(19) United States
(12) Patent Application Publication
(10) Pub. No.: US 2006/0178673 A1
(43) Pub. Date: Aug. 10, 2006

(54) Lockable slide hammer and gripping apparatus

(75) Inventor: Peter Curran, Harrogate (GB)

Correspondence Address: ARTHROCARE CORPORATION, 680 VAQUEROS AVENUE, SUNNYVALE, CA, 94085-3523, US

(73) Assignee: ArthroCare Corporation, Sunnyvale, CA (US)

(21) Appl. No.: 11/352,105

(22) Filed: Feb. 9, 2006

Related U.S. Application Data
(60) Provisional application No. 60/651,680, filed on Feb. 9, 2005.

(51) Int. Cl. A61B 17/58

(52) U.S. Cl. 606/100

(57) ABSTRACT

An apparatus comprising gripping member connected to a shaft member and in communication with a hammer member disposed on said shaft member, the hammer member including a lockable member adapted for immobilizing the hammer member on the shaft member. Advantageously, since the hammer member is lockable on the shaft and serves as a handle, and also since a wide range of instruments are attachable to gripping member, this apparatus and its use thereof eliminates the awkwardness and inefficiency inherent with prior art apparatus and procedures for inserting and retracting instruments as, for example, in inserting and retracting surgical instruments in a body in a surgical procedure.

### CROSS-REFERENCES TO RELATED APPLICATIONS

The present invention claims priority to U.S. Provisional Application No. 60/651,680, filed Feb. 9, 2005.

### BACKGROUND OF THE INVENTION

1. Field of the Invention

This invention relates generally to slide hammers and gripping apparatuses, and more specifically to a unit lockable slide hammer and a gripping apparatus useable for attaching and manipulating objects such as surgical instruments.

2. Description of the Prior Art

In an illustrative application, the prior art includes tools useable in surgical procedures for manually inserting and/or retracting surgical instruments in a patient's body. For example, in arthroscopic surgery it is often desired to use tools to assist in inserting a cannula into a joint structure to introduce and/or remove fluids from the joint, and subsequently retract the instrument. In this application, since the instruments are typically small, the instruments can be awkward to handle and manipulate. Additionally, the instruments can be troublesome to insert and retract into hard and bony parts of the body. For procedures such as these wherein the insertion and retraction are difficult, it is often necessary to use tools such as hammers to deliver a blow to another extension tool in contact with the instrument to aid in the insertion and/or retraction.

Thus in the prior art wherein a hammer and an extension tool are required for simultaneous use, the procedure is awkward and not very practical since at least two tools are being manipulated at the same time, i.e., the hammer and the extension tool. Also, where a traditional slide hammer is used, the hammer on being released after use can become a hindrance by sliding down on the shaft and getting in the line of sight of the user. Further, since a wide range of shapes and sizes of objects may be required for manipulation, it is necessary to have a corresponding range of extension tools and hammers on standby, thus incurring inefficiencies and inconvenience.

Accordingly, in view of the awkwardness, inefficiency and inconvenience in the prior art, there is a need for better tools and procedures for aid in manipulating objects, including instruments for inserting and extracting the body of a patient. It is therefore an objective of this invention to address this need.

### SUMMARY OF THE INVENTION

In accordance with one embodiment, and with reference to FIG. 1-3, the present invention comprises a unit apparatus (30) that includes a gripping member (10) connected to a shaft member (14) in communication with hammer member (16) disposed on shaft member (14), wherein hammer member (16) defines a lockable member (18) adapted for immobilizing hammer member (16) on shaft member (14).

In another embodiment, the invention comprises a surgical apparatus (30) comprising an elongate shaft member (14) having a distal end, and a proximal end; a clamp (10) arranged at said distal end, said clamp adapted to detachable secure a surgical tool; a handle member (16) slidable along said shaft member (14) between said proximal end and said distal end, said handle member (14) comprising a lock member (18) that secures the handle member (16) at a location along said shaft member (14) wherein said handle member (18), when in a locked configuration, provides a secure surface for a surgeon to manually manipulate the apparatus (30) and any tools connected thereto, and when said handle element (16) is in an unlocked configuration, provides a hammer element (16) that allows the surgeon to hit a surface of the apparatus to urge the apparatus (30) proximally or distally.

In another embodiment, the invention, with reference to FIG. 4, comprises a method of manipulating objects (400), comprising: providing an apparatus in accordance with the present invention (401) and using the apparatus (402) to manipulate objects.

In yet another embodiment, the invention comprises a method of manipulating instruments in a surgical procedure comprising: providing the present apparatus and using the apparatus for inserting and retracting instruments in a body.

Advantageously, since hammer member (16) of the present apparatus (30) is lockable on shaft member (14) and thereby can serve as a handle, and also since a wide variety of objects are attachable to gripping member (10), the apparatus of the invention (30) eliminates the awkwardness, inefficiency and inconvenience inherent with prior art apparatus and procedures.

### BRIEF DESCRIPTION OF THE DRAWINGS

FIG. 1 is a cross-section view of an embodiment of the apparatus of the present invention.

FIG. 2 is a perspective view of an embodiment of the apparatus of the invention wherein the hammer member is shown in an immobilized position on the shaft member.

FIG. 3 is a perspective view of an embodiment of the apparatus of the invention wherein an impacting member is shown in an unlocked position along the shaft member.

FIG. 4 is a block diagram of an embodiment of the invention wherein the present apparatus is provided and used for a surgical procedure.

### DETAILED DESCRIPTION OF A PREFERRED EMBODIMENT

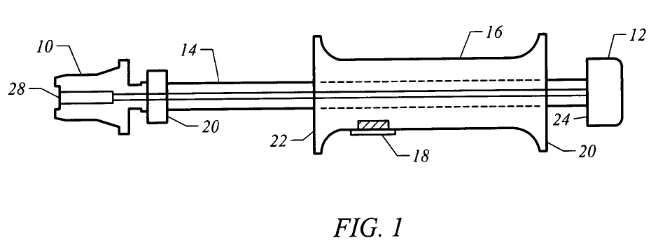

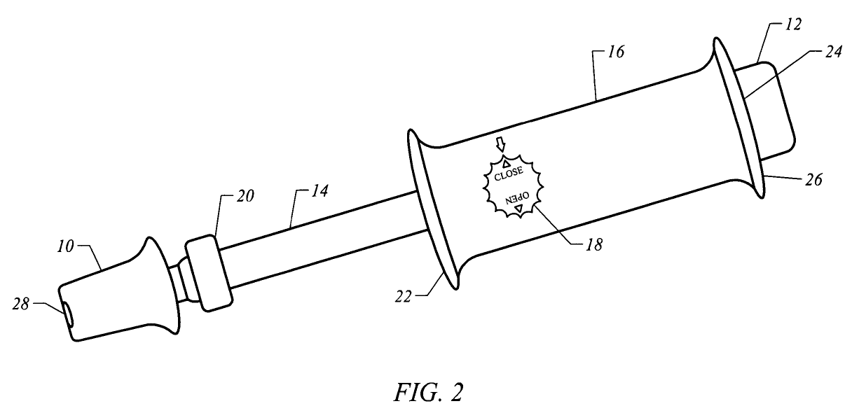

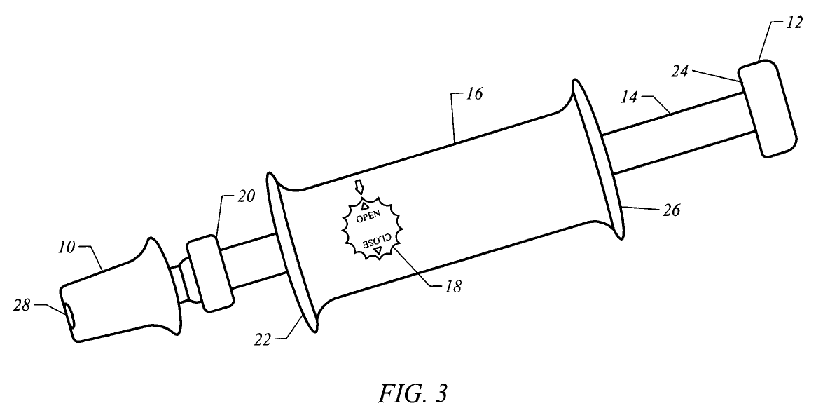

Embodiments of the present invention as illustrated FIGS. 1-4 provide for an apparatus (30) useable for manipulating a wide range of objects. In this regard, although the invention disclosed herein is described in the context of a surgical apparatus and procedure, it will be appreciated by those ordinarily skilled in the art that since the invention is adaptable in obvious ways for alternative uses, the invention is not limited to uses described herein, but encompasses apparatus and uses wherein a unit lockable slide hammer in a combination with a gripping member in accordance with this invention is used to manipulate objects.

As illustrated in the cross-section view of FIG. 1 the invention in one embodiment includes a unit apparatus (30) comprising gripping member (10) connected to shaft member (14) and in communication with hammer member (16) disposed on shaft member (14), wherein hammer member (14) includes lockable member (18) adapted for immobilizing hammer member (16) on shaft member (14).

In one embodiment, gripping member (10) comprises an orifice or a closable passageway (28) or means for receiving and gripping objects (and surgical tools) and facilitating manipulating objects. Examples of such means include a chuck operable to accommodate a wide range of objects including surgical instruments such as air-activated instruments, manually operable instruments, tunnel dilators, screw drivers, reamers, broaches and femoral locators. In use, gripping member (10) is rigidly fixed to shaft member (14), however, in one embodiment, the gripping member (10) is detachable from shaft member (14). Other embodiments of gripping member include attachable/detachable claws and their equivalents.

Shaft member (14) as illustrated in FIG. 1, in one embodiment comprises a rigid member adaptable for receiving and supporting gripping member (10), hammer member (16) and stop member (12). In one embodiment, shaft member (14) includes threadable proximal and distal end sections to receive gripping member (10) and stop member (12) respectively. As will be obvious to one ordinarily skilled in the art, many variations in attaching gripping member (10) and stop member (12) can be used as are materials to form shaft member (14). In use, shaft member (14), in combination with gripping member (10), hammer member (16) and stop member (12) is maneuverable as a unit to grasp and manipulate objects including surgical instruments.

Hammer member (16) in one embodiment is slidably mounted on shaft member (14) and comprises a body useable to deliver a blow to gripping member (10) at surfaces (20, 22) and stop member (12) at surfaces (24, 26). In one embodiment, hammer member (16) is adapted for delivering a blow to gripping member (10), and includes a locking member, or fixing means for immobilizing and releasing hammer member (16) on shaft member (14). In another embodiment hammer member (16) is detachable from shaft member (14). In yet another embodiment, hammer member (16) is slidably attached to shaft member (14).

In one embodiment the fixing means comprises a thumbscrew defined by hammer member (16). In one embodiment, lockable member (18) is designed with gripping elements for ease of use in securing handle member (16) to shaft member (14) during surgical operations

Stop member (12) is an element disposed distally from gripping member (10) on shaft member (14). Structurally, stop member provides a surface (24) for receiving a blow from hammer member (16) to aid, for example, in extracting a surgical instrument stuck in a bone and is being gripped in gripping member (10).

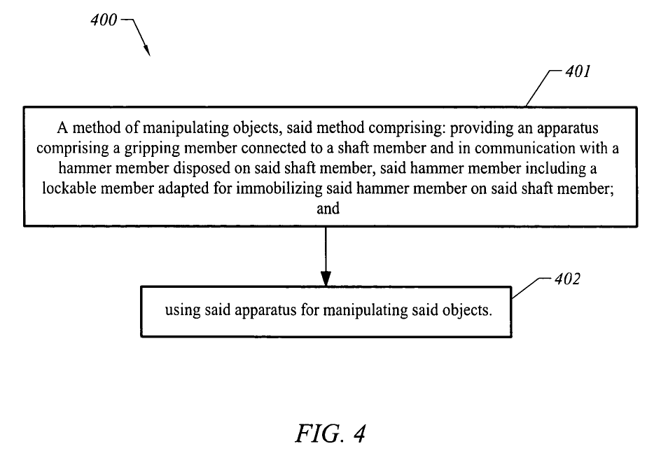

In use the invention in one embodiment as shown in FIG. 4 comprises a method of manipulating objects (400), comprising the step of providing an apparatus (401) wherein gripping member is connected to a shaft member (14) and in communication with hammer member (16) disposed on shaft member (14), and wherein hammer member (16) includes lockable member (18) adapted for immobilizing hammer member (16) on shaft member (14); and the step of using the apparatus (30) for inserting and retracting instruments.

In another application, the invention comprises a method performing a surgical procedure, comprising the step of using an apparatus comprising a gripping member (10) connected to shaft member (14) and in communication with hammer member (16) disposed on shaft member (14), wherein hammer member (16) includes lockable member (18) adapted for immobilizing the hammer member (16) on shaft member (14). Locking member (18) may be, for example, a thumbscrew.

Optionally the method further includes attaching at least one surgical instrument to gripping member (10), and operating hammer member (16) to deliver blows to gripping member (10). The method also includes the optional step of releasing surgical instruments (not shown) from gripping member (10); and immobilizing hammer member (16) on shaft member (14). Additionally, the method includes optionally manipulating the surgical instruments by removing the surgical instruments by attaching the instrument-gripping member (10) to the surgical instruments; and operating hammer member (16) to deliver blows to stop or end member (12). The method further includes the step of releasing the surgical instruments from gripping (10) member, and immobilizing the hammer member (16) on shaft member (14).

In another embodiment the method of the invention further comprises proximally urging the hammer member (16) against an opposing surface to retract the surgical instrument the target location. In one embodiment the target location is a bone tunnel. In a further embodiment, the method the surgical instrument is one instrument selected from the group consisting of a reamer, drill, dilator, broach and cannula.

Examples of surgical procedures that may be carried out with the apparatus, depending on the tool being held, include creating bone tunnels for articular cruciate reconstructions, rotator cuff repair, and compacting, drilling, and removing hard and soft tissue in various other surgical procedures.

While the invention is described in the context of an apparatus useable for inserting and retracting instruments in a surgical procedure, it will be appreciated by one ordinarily skilled in the art that the invention can also be practiced with obvious modifications for wide range of equivalent applications wherein a lockable sliding hammer is useable in conjunction with a gripping member for manipulating objects, including surgical instruments. Thus the scope of the invention should not be limited to the embodiments as described herein, but is limited only by the appended claims and their legal equivalents.

What is claimed is:

1. An apparatus comprising:
a gripping member connected to a shaft member and in communication with a hammer member disposed on said shaft member, said hammer member including a lockable member adapted for immobilizing said hammer member on said shaft member.

2. The apparatus of claim 1, wherein said gripping member comprises means for receiving and gripping at least one surgical instrument.

3. The apparatus of claim 1, wherein said gripping member is detachable from said shaft member.

4. The apparatus of claim 1, wherein said gripping member comprises a chuck member.

5. The apparatus of claim 2, wherein said surgical instrument is selected from an air-activated instrument, a manually operable instrument, a tunnel dilator, a screw driver, a reamer, a broach and a femoral locator.

6. The apparatus of claim 1, wherein said hammer member is adapted for delivering a blow to said gripping member.

7. The apparatus of claim 1, wherein said hammer member comprises a fixing means for immobilizing and releasing said hammer member on said shaft member.

8. The apparatus of claim 1, wherein said hammer member is detachable from said shaft member.

9. The apparatus of claim 7, wherein said fixing means comprises a lockable pin member.

10. The apparatus of claim 7, wherein said fixing means is operable to lock and release said hammer member to said shaft member during use of said surgical apparatus.

11. The apparatus of claim 1, wherein said hammer member is adapted for use as a handle for manipulating said gripping member during use of said surgical apparatus.

12. The apparatus of claim 1, wherein shaft member comprises a stop member disposed distally from said gripping member on said shaft member.

13. The apparatus of claim 12, wherein said hammer member is adapted for delivering a blow to said stop member.

14. The apparatus of claim 12, wherein said stop member is detachable from said shaft member.

15. A method of manipulating objects, said method comprising: providing an apparatus comprising a gripping member connected to a shaft member and in communication with a hammer member disposed on said shaft member, said hammer member including a lockable member adapted for immobilizing said hammer member on said shaft member.

16. The method of claim 15, comprising using said apparatus for inserting and retracting instrument objects.

17. A method for performing a surgical procedure, said method comprising: using an apparatus comprising a gripping member connected to a shaft member and in communication with a hammer member slidably disposed along said shaft member, said hammer member including a lockable member adapted for immobilizing said hammer member on said shaft member.

18. The method of claim 17, further comprises:
attaching at least one surgical instrument to said gripping member; and
operating said hammer member to deliver blows to said gripping member.

19. The method of claim 18, further including:
immobilizing said hammer member on said shaft member.

20. The method of claim 17 comprising:
attaching a surgical instrument to said gripping member, said gripping member arranged at a distal end of said apparatus; and
inserting said surgical instrument into a target location within a human.

21. The method of claim 20, further comprising:
proximally urging said hammer member against an opposing surface to retract said surgical instrument from said target location.

22. The method of claim 21 wherein said target location is a bone tunnel.

23. The method of claim 22 wherein said surgical instrument is one instrument selected from the group consisting of a reamer, drill, dilator, broach and cannula.

24. A surgical apparatus comprising
an elongate shaft having a distal end, and a proximal end;
a clamp arranged at said distal end, said clamp adapted to be detachable and to secure a surgical tool;
a handle slidable along said shaft between said proximal end and said distal end, said handle comprising a lock that secures the handle at a location along said shaft wherein said handle, when in a locked configuration, provides a secure surface for a surgeon to manually manipulate the apparatus and any tools connected thereto, and when said handle is in an unlocked configuration, provides a hammer that allows the surgeon to hit a surface of the apparatus to urge the apparatus proximally or distally.
